SEQUENCE LISTING (1 sequence)

SEQ ID NO: 1 - protein, 300 aa, Homo sapiens
MRIAVICFCLLGITCAIPVKQADSGSSEEKQLYNKYPDAVATWLNPDPSQKQNLLAPQTLPSKSNESHDHMDDMDDEDDDDHVDSQDSIDSNDSDDVDDTDDSHQSDESHHSDESDELVTDFPTDLPATEVFTPVVPTVDTYDGRGDSVVYGLRSKSKKFRRPDIQYPDATDEDITSHMESEELNGAYKAIPVAQDLNAPSDWDSRGKDSYETSQLDDQSAETHSHKQSRLYKRKANDESNEHSDVIDSQELSKVSREFHSHEFHSHEDMLVVDPKSKEEDKHLKFRISHELDSASSEVN

(19) United States
(12) Patent Application Publication
(10) Pub. No.: US 2010/0086525 A1
(43) Pub. Date: Apr. 8, 2010

(54) Compositions and Methods for Altering Pancreas or Liver Function

(76) Inventors: Kenneth S. Zaret, Elkins Park, PA (US); Yasushige Kashima, Tochigi (JP)

Correspondence Address: LICATA & TYRRELL P.C., 66 E. MAIN STREET, MARLTON, NJ, 08053, US

(21) Appl. No.: 12/598,439

(22) PCT Filed: Apr. 18, 2008
(86) PCT No.: PCT/US08/60730

§ 371 Date: Nov. 2, 2009

Related U.S. Application Data
(60) Provisional application No. 60/915,728, filed on May 3, 2007.

(51) Int. Cl. A61K 38/16; C12N 5/00; C12N 5/071; A61K 35/407; A61K 35/39; A61P 1/16; A61P 1/18

(52) U.S. Cl. 424/93.7; 435/377; 514/12; 435/370

(57) ABSTRACT

Methods for altering pancreatic and liver cell function are provided, wherein the compositions and methods are based on use of osteopontin or on altering the activity of osteopontin.

### BACKGROUND OF THE INVENTION

Osteopontin (OPN) is a highly acidic secreted phosphoprotein. OPN is an integrin- and calcium-binding protein that has been localized in mineralized tissues (bone), epithelial cells, activated cells of the immune system, bladder smooth muscle cells, kidney, ovary, and uterus (Brown, L. F. et al. 1992. Mol. Biol. Cell 3:1169-1180; Arafat, H. A. et al. 2007. Endocrinology 148:575-584). OPN has been associated with a variety of functions including cell adhesion and migration, inflammatory reactions, and apoptosis (Kolb, A. et al. 2005. Cancer Biol. Ther. 4:740-746). The human OPN nucleic acid and amino acid sequence are known and may be found in the National Center for Biological Information (NCBI) UniGene data base under accession number Hs. 313.

The precise roles that OPN may play in normal tissue development and maintenance, as well as in embryogenesis and fetal development are not known at this time. Some of the effects of OPN appear to be mediated by interaction of OPN with integrin molecules via its RGD (arginine-glycine-aspartic acid) amino acid sequence. Studies in mouse macrophages, ventricular myocytes, cardiac microvascular endothelial cells, mouse kidney epithelial cells, and rat pancreatic islet cells have linked OPN activity to regulation of nitric oxide production and signaling (Arafat, H. A. et al. 2007. Endocrinology 148:575-584).

Increased expression of OPN has been observed in the pancreas in chronic pancreatitis (Nakamura, M. et al. 2002. Pancreas 25:182-187), in pancreatic cancer (Sedivy, R. et al. 2005. Virchows Arch. 446:41-45), and in a rat model of diabetes (Katakam, A. K. et al. 2005. J. Endocrinol. 187:237-247). OPN has been shown to influence the invasiveness of pancreatic cancer cells (Kolb, A. et al. 2005. Cancer Biol. Ther. 4:740-746). Recently, OPN deficiency was also been shown to alter the pancreatic cytokine profile (TNF-α, IFN-γ, IL-10, IL-4) in a mouse model of diabetes, with increases in OPN expression in the pancreas seen following induction of diabetes and OPN deficiency characterized by less islet infiltration and apoptosis as compared to wild-type diabetic mice (Arafat, H. A. et al. 2006. Exp. Clin. Endocrinol. Diabetes 114:555-562). OPN has also been reported to act as a marker of undifferentiated pancreatic precursors and pancreatic ductal tissue in mice (Kilic, G. et al. 2006. Develop. Dynam. 235:1659-1667). In this study, the authors reported that there was a specific, dynamic profile of OPN expression in embryonic pancreatic tissues that suggested participation of the protein in cell migration and/or cell-cell interactions, whereas OPN-deficient pancreata showed no obvious alterations in morphology or differentiation.

A variety of patent applications and issued patents describe methods of treating diseases or conditions by altering activity of OPN. WO 2003/0044862 teaches OPN as a marker for tumor hypoxia in head and neck cancer. WO 2003/077948 describes administering OPN to treat myeloma. WO 2003/087766 teaches inhibiting metastases of hepatocellular carcinoma by decreasing OPN activity. WO 2003/100007 discusses enhancing immune responses by administering OPN. WO 2004/0235720 teaches methods of preventing or treating neurologic diseases by administering OPN, where the neurologic diseases listed include traumatic nerve injury, stroke, demyelinating diseases, neuropathies, and neurodegenerative disorders. WO 2005/009468 describes a remedy for cartilage diseases that involves inhibiting the activity or expression of OPN. WO 2005/049083 discusses use of antibodies to OPN as a treatment for tendon and/or ligament deterioration. WO 2005/053628 teaches a method for reducing plaque growth on teeth and treatment of dental disease by administering OPN. WO 2006/043954 describes treating tumors by administering antibodies to OPN, specifically breast and ovarian tumors. U.S. Pat. No. 5,695,761 teaches methods of inhibiting inflammation mediated by nitric oxide by administering OPN. U.S. Pat. No. 6,458,590 discloses a method of treating restenosis following vascular surgery by inhibiting activity of OPN. U.S. Pat. No. 6,551,990 teaches a method of inhibiting ectopic calcification by administering OPN.

Due to the biological importance of OPN in a variety of tissues and pathologies, the OPN gene has been suggested to be an important target for embryonic stem cell manipulation (U.S. Pat. No. 6,414,219). It has now been found that OPN acts as a signaling molecule secreted from endothelial cells and is capable of inducing pancreatic and liver cell differentiation in embryonic cells, thereby having the ability to alter pancreatic and liver cell function.

### SUMMARY OF THE INVENTION

An object of the present invention is a method for inducing differentiation of an endodermal cell or a progenitor cell into a pancreatic or liver cell which comprises contacting an endodermal cell or a progenitor cell with an effective amount of osteopontin thereby inducing differentiation of the endodermal cell or the progenitor cell into a pancreatic or liver cell. Also contemplated by the present invention is a method wherein the cell is a partially differentiated liver or pancreatic progenitor cell.

Another object of the present invention is a method for altering liver cell function which comprises contacting a liver cell with an effective amount of osteopontin, wherein contact of the cell with osteopontin results in an alteration in the function of the liver cell.

Another object of the present invention is a method for altering liver cell function which comprises contacting a liver cell with an effective amount of a composition comprising an osteopontin protein, an osteopontin antibody, an osteopontin mimetic, an osteopontin agonist, an osteopontin antagonist, a mutated osteopontin protein, or an osteopontin variant or fragment thereof, formulated in a pharmaceutically acceptable vehicle, wherein contact of the cell with said composition results in an alteration in the function of the liver cell.

Another object of the present invention is a method for preventing or treating a disease of the liver in a patient comprising administering to a patient a therapeutically effective amount of a composition comprising osteopontin formulated in a pharmaceutically acceptable vehicle, wherein administration of said composition results in prevention or treatment of a disease of the liver.

Another object of the present invention is a method for preventing or treating a disease of the liver in a patient which comprises administering to a patient a therapeutically effective amount of a composition comprising an osteopontin protein, an osteopontin antibody, an osteopontin mimetic, an osteopontin agonist, an osteopontin antagonist, a mutated osteopontin protein, or an osteopontin variant or fragment thereof, formulated in a pharmaceutically acceptable vehicle, wherein administration of the composition results in prevention or treatment of a disease of the liver.

Yet another object of the present invention is a method of restoring function of a damaged liver tissue which comprises contacting a damaged liver tissue with an effective amount of osteopontin, wherein contact of the damaged tissue with osteopontin results in a restoration of function of the damaged tissue.

Finally, other objects of the present invention include isolated pancreatic or liver cells differentiated by the method of the present invention as well as pharmaceutical compositions which comprise isolated pancreatic or liver cells differentiated by the method of the present invention and a pharmaceutically acceptable vehicle.

### DESCRIPTION OF THE DRAWINGS

FIG. 1 depicts the growth of tissue explants of dorsal pancreatic endoderm, both wild-type flk-1+ and flk-1^{-/-}, when co-cultured with various types of cells. FIGS. 1A through 1D depict PECAM-CD31 immunohistochemistry. A CD-31 vascular network is reconstructed by the co-culture of eEND2 cells with the flk-1^{-/-} explants.

FIG. 2 depicts the results of experiments with RT-PCR cycle step analysis. FIGS. 2A and 2B respectively show results where eEND2 or 3T3 cells are co-cultured with flk-1^{-/-} dorsal pancreatic endoderm explants. Only in the co-culture with eEND2 is the expression of the ptf1a gene detected.

FIG. 3 depicts results from a more sensitive qRT-PCR assay of gene expression that normalized signals to actin mRNA. Conditioned medium from various endothelial cell lines, but not the control cell lines, induced the expression of several known explant tissue differentiation genes (ptf1a^{p48}, pdk-1, Isl-1).

FIG. 4 depicts the growth of dorsal pancreatic endoderm explants in the presence of conditioned medium from the endothelial cell lines.

FIG. 5 depicts the results of experiments using eEND2 conditioned medium. Using a 10K cutoff filter in an ultra-centrifugation process to separate components of the conditioned medium, ptf1a and pdx activity was discovered in the 10K retentate and not the 10K flow-through.

FIG. 6 depicts results of experiments using eEND2 conditioned medium where ptf1a activity was measured after incubating retenate for 10 minutes at 60° C. or by boiling or trypsin treatment. Induction of ptf1a was inhibited by boiling or trypsin treatment, consistent with the induction being mediated by a protein.

FIG. 7 depicts results of experiments using eEND2 conditioned medium where ptf1a activity was measured after passage through either 50K or 100K filters. The ptf1a induction activity was successively retained on the filters in 50K and 100K ultracentrifugation assays, indicating that it depends upon a large molecule or complex.

FIG. 8 depicts the results of experiments using eEND2 conditioned medium where ultra-filtration was performed in the presence of 25% acetonitrile and 0.1 M glycine (pH 2.3) to denature proteins, and the retentate was then re-natured, followed by measurement of ptf1a activity. The retentate retained 60% of ptf1a induction activity and was not enhanced by combination with renatured flow-through material, indicating that the ptf1a induction activity is not due to a small molecule bound to a large molecule.

FIG. 9 depicts results or experiments using eEND2 conditioned medium where a 10K retenate was sequentially fractionated at 50K and 100K, and the 100K retentate was fractionated by anion-exchange fast protein liquid chromatography (FPLC), followed by measurement of ptf1a activity in collected fractions. The ptf1a inducing activity was in the 100K/FPLC-Fr3 fraction.

FIG. 10 depicts results of a SDS-PAGE analysis of the active and inactive FPLC fractions from the eEND2 100K retenate and the inactive Fr3 from the 3T3 100K retentate.

FIG. 11 depicts the amino acid sequence for osteopontin (SEQ ID NO:1).

FIG. 12 depicts results showing that osteopontin is sufficient to induce endodermal cell differentiation. FIG. 12A depicts results from a qRT-PCR assay of gene expression that normalized signals to actin mRNA. It shows that purified proteins at the amounts indicated induced the expression of several known explant tissue differentiation genes (ptf1a^{p48}, pdk-1, Isl-1, ngn-3). FIG. 12B is a western blot showing that osteopontin was detected in the conditioned medium from the endothelial cell lines and 3T3 control cells, but not in that from 293T cells. FIG. 12C is a western blot showing that osteopontin is not present in cell lysates. FIG. 12D depicts results showing that antibodies to osteopontin inhibit the ptf1a inducing activity of osteopontin in conditioned medium.

FIG. 13 depicts the effects of treatment with osteopontin on induction of albumin and alpha-fetoprotein expression in liver bud explants from flk-1^{-/-} embryos. FIG. 13A depicts the effects of osteopontin on albumin while FIG. 13B depicts the effects of osteopontin on alpha-fetoprotein. These data show that osteopontin treatment dramatically enhances liver differentiation of progenitor cells.

### DETAILED DESCRIPTION OF THE INVENTION

It has become evident that signaling from endothelial cells can promote the differentiation, growth and homeostasis of diverse tissues outside the cardiovascular system, independent of endothelial cell function as a conduit for substances in the bloodstream. Despite the diversity of endothelial cell signaling in gut organ development, the identity of relevant signaling molecules has been unclear. Using a combination of genetics, embryology, biochemical purification and proteomics, individual signaling molecules have been identified that are secreted from endothelial cell lines and induce aspects of pancreatic and liver cell differentiation of endoderm cells. One such molecule that has been identified is osteopontin. Therefore, the present invention includes methods for modulation of pancreatic cell and liver cell differentiation that involve modulating activity of osteopontin.

Specific applications of the present invention include use of osteopontin, or compounds that modulate osteopontin activity, to affect pancreatic and liver cell differentiation and ultimately pancreatic or liver cell function. In the context of the present invention, compounds that lead to “modulation of osteopontin activity” include compounds that lead to either an increase or a decrease in the activity of osteopontin in pancreatic or liver cells. Such compounds contemplated by the present invention include compounds that act as osteopontin agonists or antagonists, antibodies to osteopontin, osteopontin mimetics, mutated osteopontin proteins, or osteopontin variants or fragments. Use of compounds that modulate osteopontin activity, or use of osteopontin itself, in methods of the present invention will lead to alterations in the function of pancreatic or liver cell function based on the finding that osteopontin has the ability to alter pancreatic and liver cell differentiation activity.

Reciprocal signaling between endothelial cells and smooth muscle cells, and between endothelial cells and cardiac muscle cells, is necessary for proper development of the cardiovascular system (Risau. 1997. Nature 386:671-674). Such signaling involves distinct classes of ligand-receptor interactions, which in some cases also promote neuronal development (Carmeliet and Tessier-Lavigne. 2005. Nature 436:193-200). Only recently, however, has it been appreciated that endothelial cells signal directly to epithelial cells in gut organs, such as during liver, pancreas and thyroid development as well as during regenerative responses to tissue damage (Cleaver and Melton. 2003. Nat. Med. 9:661-668; Red-Horse et al. 2007. Dev. Cell 12:181-194). Direct signaling refers to signaling from endothelial cells to other cell types, and is not due to endothelial cell function as a conduit for components in the bloodstream. For example, flk-1^{-/-} mouse embryos, which are genetically deficient in endothelial cells (Shalaby et al. 1995. Nature 376:62-66), exhibit major defects in pancreatic endoderm differentiation and liver bud growth (Matsumoto et al. 2001. Science 294:559-563; Lemmert et al. 2001. Science 294:564-567; Yoshitomi and Zaret. 2004. Development 131:807-817). Co-culture of wild-type aortae with pancreatic endoderm tissue from flk-1^{-/-}, in the absence of blood flow, is sufficient to restore differentiation (Yoshitomi and Zaret. 2004. Development 131:807-817). Similarly, liver bud growth in vitro is markedly enhanced by resident endothelial cells (Matsumoto et al. 2001. Science 294:559-563). Hepatocyte growth factor (HGF) produced from endothelial cells can promote regeneration after liver cell damage (LeCouter et al. 2003. Science 299:890-893) and extracellular matrix proteins produced from endothelial cells can help maintain adult pancreatic islet function (Nikolova et al. 2006. Nat. Prod. Res. 20:103-106). Given the emerging contexts of direct endothelial cell signaling in gut organ biology and organogenesis, and the potential for applying the knowledge to directed cell differentiation and regenerative medicine, the identity of endothelial proteins that promote early pancreas and liver organogenesis was sought.

Experiments were performed to isolate and identify putative endothelial cell signaling molecules. Four endothelial cell lines were used in the experiments (eEND2, bEND-3, HUVEC and HUAEC) as well as two control cell lines (3T3 and 293T). The cell lines were screened for the existence of compounds that had the ability to restore parameters of gut organ development in flk-1^{-/-} tissue explant assays.

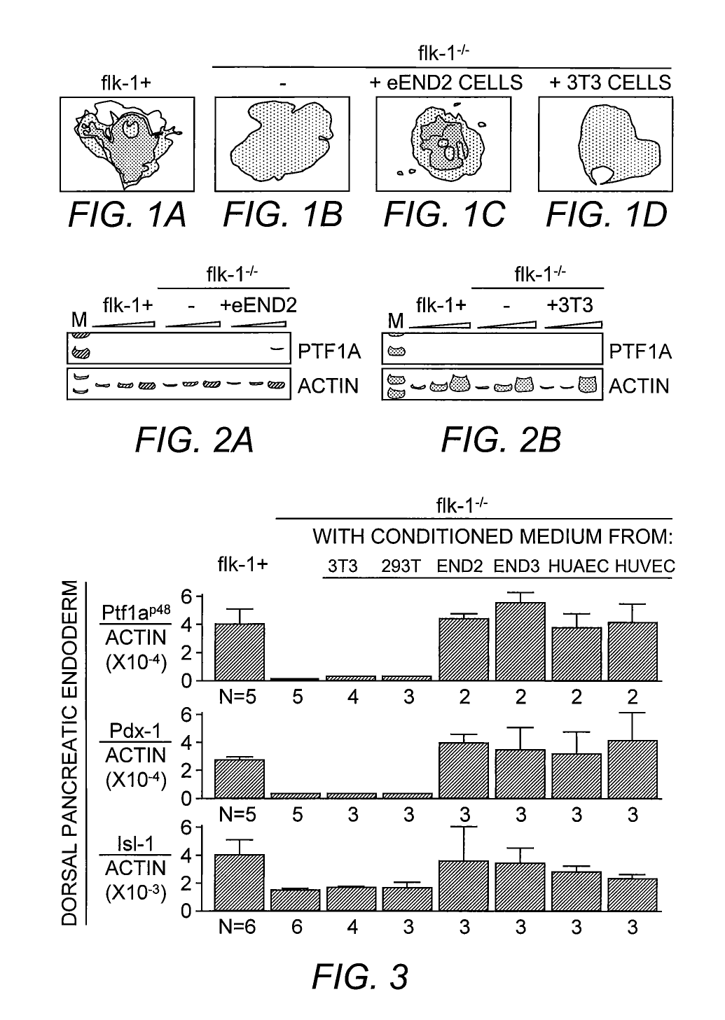

Dorsal pancreatic endoderm and liver buds were microdissected from mouse embryos of flk-1^{+/-} and flk-1^{-/-} genotypes at nine days gestation (E9.0) and cultured on a Transwell membrane at the air-liquid interface as previously described (Matsumoto et al. 2001. Science 294:559-563; Yoshitomi and Zaret. 2004. Development 131:807-817), in the presence and absence of cell lines previously seeded onto the membrane. Results showed that eEND2 cells, a permanent mouse endothelial cell line, were able to integrate into the flk-1^{-/-} tissues and generate a CD-31 positive network of cells that resembled the native vascular network generated in wild-type tissue explants (FIG. 1A). Similar results were obtained with co-culture of bEND-3 cells and HUAEC endothelial cell lines. In contrast, 3T3 control cells, a non-endothelial cell line, were not able to integrate into the flk-1^{-/-} tissues, nor did the control cells result in generation of a vascular network that resembled wild-type tissue explants (FIG. 1). HUVEC cell co-culture with flk-1^{-/-} tissues produced results similar to the 3T3 control cells. The presence of endothelial cells did not markedly affect the overall growth of epithelial cells in the tissue explant (FIGS. 1B and 1C). These data demonstrated that co-culture of endothelial cells with the endothelial-cell deficient flk-1^{-/-} tissue affected the development of the tissues, implicating substances present in or produced by the endothelial cells in the altered cellular physiology.

Experiments were then performed to identify gene expression changes in the co-cultured cells as a first step in identifying the putative endothelial cell signaling molecules. RT-PCR cycle step analysis showed that co-culture of eEND2 cells with flk-1^{-/-} dorsal pancreatic endoderm explants dramatically enhanced expression of the ptf1a^{p48} gene (FIG. 2A), whereas co-culture of the explants with 3T3 cells failed to enhance expression of this gene (FIG. 2B). Expression of ptf1a^{p48} was not detected in flk⁺ explants (FIG. 2) The ptf1a^{p48} gene encodes a transcription factor that is induced in wild-type embryos at very low levels at E9.0 (Yoshitomi and Zaret. 2004. Development 131:807-817), yet is crucial for subsequent pancreatic differentiation (Krapp et al. 1998. Genes Dev. 12:3752-3763; Kwaguichi et al. 2002. Nat. Genet. 32:128-134). Further, it has been shown that ptf1a^{p48} expression in the dorsal pancreatic endoderm, but not the expression of various other pancreatic regulatory factors, is critically dependent upon cell interactions with the aortic endothelium (Yoshitomi and Zaret. 2004. Development 131:807-817). Considered together, these results demonstrated that endothelial cell lines were able to complement the genetic deficiency of endogenous vascular cells in flk-1^{-/-} tissue explants and promote an early step of pancreatic differentiation.

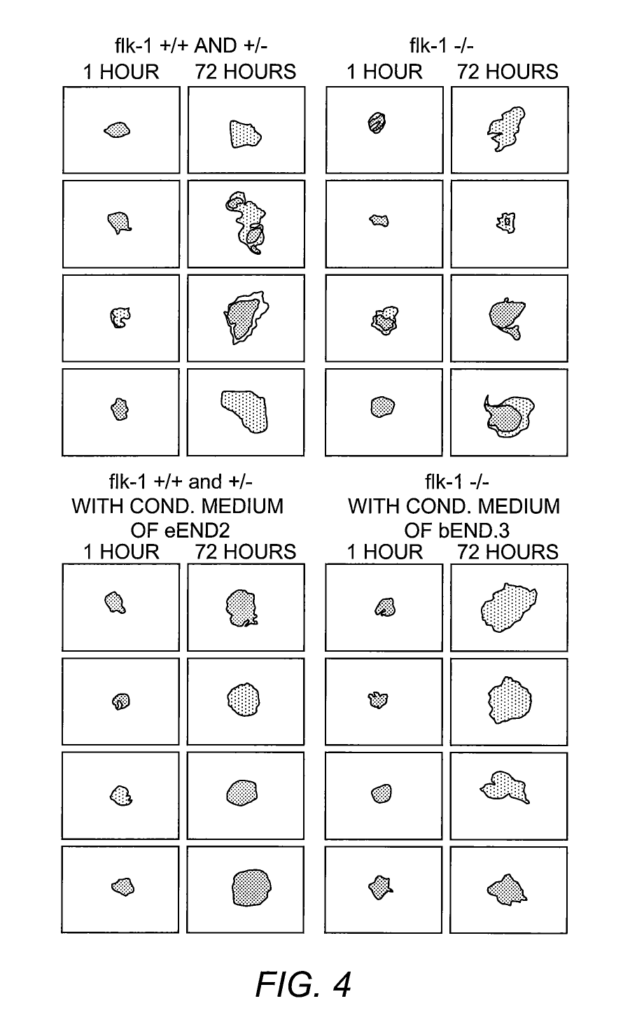

To define the nature of the interactions between endothelial cells and tissue explants as involving putative signaling molecules as opposed to some other type of cell-cell interaction, studies were performed with medium collected from endothelial cell lines. A more sensitive qRT-PCR assay of gene expression was developed that normalized signals to actin mRNA. Conditioned medium was obtained by culturing the cell lines in plastic dishes to near confluence, washing the cells extensively, cultivating the cells with basal (DMEM) medium without serum or added growth factors for two days, and collecting and filtering the medium. Results showed that conditioned medium from the endothelial cell lines, but not the control cell lines, was sufficient to induce the expression of several known explant tissue differentiation genes (FIG. 3). The expression of such genes was markedly enhanced (near levels of wild-type explants, flk-1⁺) only when conditioned medium of endothelial cell lines was used. Moreover, the conditioned medium from the endothelial cell lines had this effect without affecting overt explant growth and replication rate (FIG. 4). A 1:1 mix (approximately 50 micrograms) of conditioned medium from all four endothelial cell lines, but none of the control cell lines, combined with normal culture medium (Yoshitomi and Zaret. 2004. Development 131:807-817) restored expression of ptf1a^{p48} in flk-1^{-/-} dorsal pancreatic endoderm explants (FIG. 3, top panel) Expression of ptf1a^{p48} is normally induced in cells that are positive for the PDX-1 homeobox transcription factor (Kawaguichi et al. 2002. Nat. Genet. 32:128-134; Chiang and Melton. 2003. Dev. Cell 4:383-393; Yoshitomi and Zaret. 2004. Development 131:807-817). Results also showed that the endothelial conditioned medium also restored pdx-1 expression to normal levels (FIG. 3, middle panel). Expression of Isl-1, which is primarily observed in the pancreatic mesenchyme at the E9.0 stage (Ahlgren et al. 1997. Nature 385:257-260), was not markedly affected by the presence or the endothelial conditioned medium (FIG. 3, bottom panel). Therefore, proteins released or secreted from endothelial cell line cultures (putative endothelial signaling molecules) were sufficient to complement a deficiency in endothelial inductive activity in flk-1^{-/-} tissue explants.

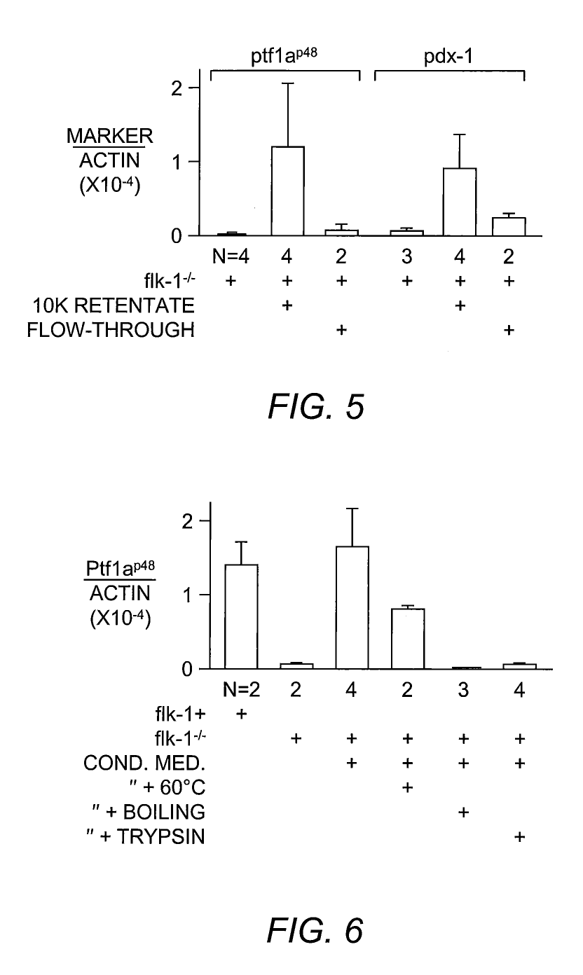

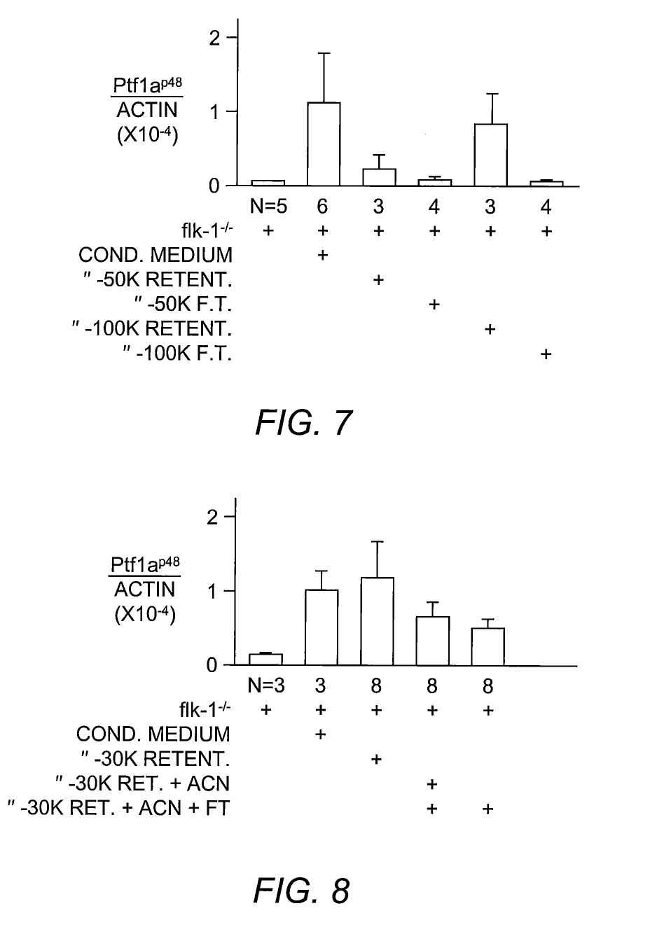

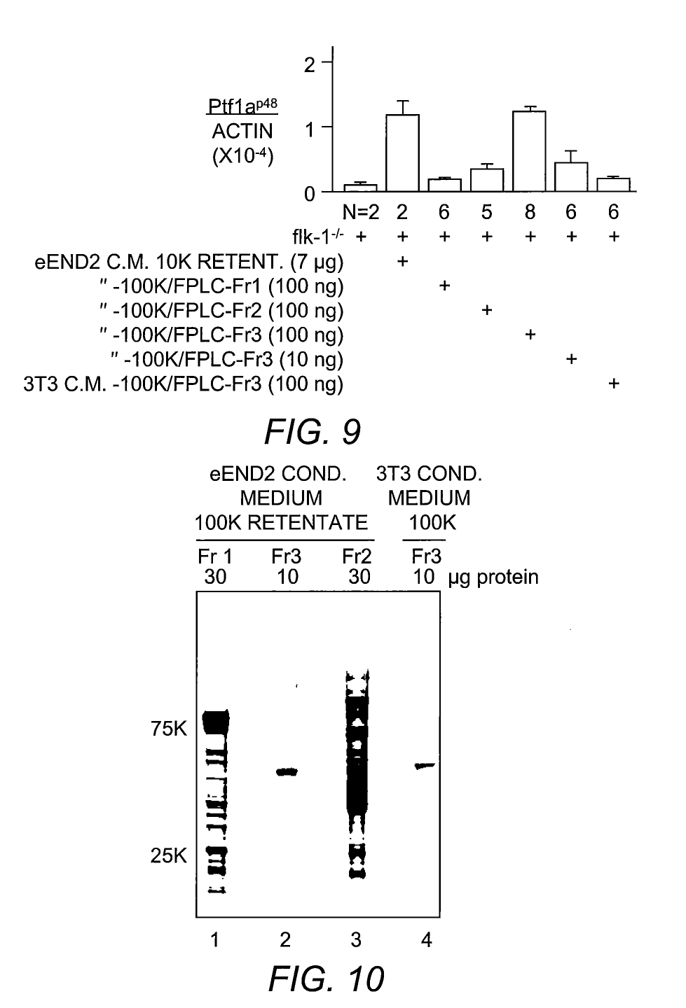

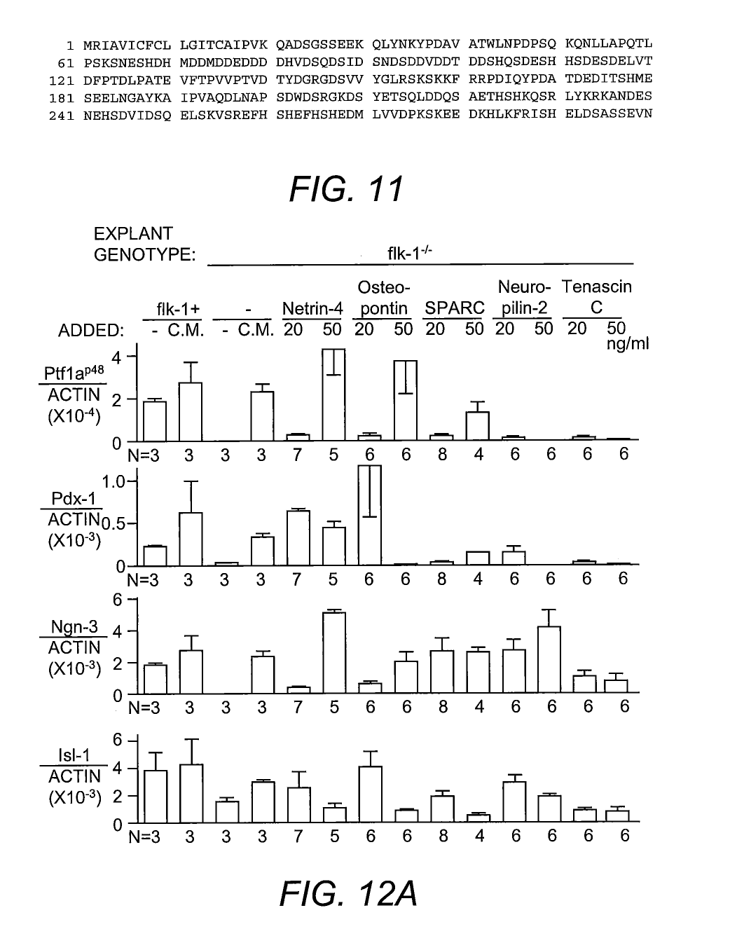

With these results, experiments were undertaken to further characterize the endothelial signaling molecules that were present in the conditioned medium. The experiments were performed using eEND2 conditioned medium. Using a 10K cutoff filter in an ultra-centrifugation process to separate components of the conditioned medium, it was shown that ptf1a and pdx induction activity was absent in the flow-through but concentrated 25-fold in the retentate (FIG. 5). These data indicated that the active molecules were not free, small molecules. Additional experiments showed that the ptf1a induction activity was partially impaired by incubating the retenate for 10 minutes at 60° C. and completely impaired by boiling or trypsin treatment (FIGS. 6 and 7). Ultra-filtration was then performed in the presence of 25% acetonitrile and 0.1 M glycine (pH 2.3) to denature proteins, and the retentate was then re-natured; the retentate retained 60% of ptf1a induction activity and was not enhanced by combination with re-natured flow-through material (FIG. 8). These results indicated that the ptf1a induction activity was not due to a small molecule bound to a large molecule. Further experiments showed that when a 10K retentate was sequentially fractionated at 50K and 100K, and the 100K retenate was fractionated by anion-exchange fast protein liquid chromatography (FPLC), ptf1a induction activity was reproducibly recovered in Fraction-3 (FIG. 9; Fr3), which represented 5% of the input FPLC material. A comparable fraction of 3T3 cell conditioned medium lacked activity (FIG. 9). Based on the fact that 100 ng of the eEND2 100K/FPLC-Fr3 was able to completely restore ptf1a expression in flk-1^{-/-} dorsal pancreatic endoderm explants, but a 10 ng sample exhibited only partial activity (FIG. 9), it can be concluded that the activity was enriched over 500-fold from the conditioned medium.

Given the lower complexity of the eEND2, 100K/FPLC-Fr3, four analyses of each were performed with tryptic digests of the 100K/FPLC-Fr3 fractions of eEND2 and 3T3 cells by automated microcapillary liquid chromatography-tandem mass spectrometry (LC-MS/MS), and one analysis one analysis of each was performed by multidimensional protein identification technology (MudPIT) with LC-MS/MS. Two analyses each of tryptic digests of the eEND2, 100K/FPLC-Fr1 and -Fr2 fractions were performed by MudPIT/LC-MS/MS. In addition, spots were excised from the eEND2 100K/FPLC-Fr3 lane and LC-MS/MS was performed. Protein identifications from the diverse approaches were compared and a list of candidate signaling proteins enriched in the active fraction was compiled (Table 1).

TABLE 1
Number of Unique Peptides Identified, Each Experiment

| LC-MS/MS and MudPIT | | | | | | | | | | | | | | | |
|---|---|---|---|---|---|---|---|---|---|---|---|---|---|---|---|
| | Exp't: | | | | | | | | | | | | | | 1D |
| | Dec. 18, 2006 | | | Dec. 23, 2006 | | | Jan. 03, 2007 | | | | Jan. 11, 2007 | | | | gel |
| | Cell Source: | | | | | | | | | | | | | | bands |
| | eEND2 | | 3T3 | eEND2 | | 3T3 | eEND2 | | | 3T3 | eEND2 | | | 3T3 | eEND2 |
| | FPLC Fraction | | | | | | | | | | | | | | |
| Protein/I.D.: | 2 | 3 | 3 | 2 | 3 | 3 | 1 | 2 | 3 | 3 | 1 | 2 | 3 | 3 | 3 |
| Netrin-4 | 0 | 0 | 0 | 0 | 0 | 0 | 0 | 0 | 1 | 0 | 0 | 0 | 3 | 0 | 0 |
| IPI00119840.1 | | | | | | | | | | | | | | | |
| Osteopontin | 1 | 1 | 1 | 1 | 1 | 1 | 0 | 0 | 1 | 0 | 0 | 0 | 1 | 0 | 0 |
| IPI00309133.6 | | | | | | | | | | | | | | | |
| SPARC | 0 | 9 | 0 | 0 | 11 | 0 | 0 | 11 | 6 | 0 | 0 | 11 | 7 | 0 | 3 |
| IPI00126343.1 | | | | | | | | | | | | | | | |
| Neuropilin-2 | 0 | 1 | 0 | 0 | 2 | 0 | 0 | 1 | 0 | 0 | 0 | 0 | 1 | 0 | 14 |
| IPO00129911.1 | | | | | | | | | | | | | | | |
| Tenascin C | 0 | 28 | 0 | 0 | 36 | 0 | 0 | 10 | 15 | 0 | 0 | 10 | 24 | 0 | 114 |
| IPI00403938.1 | | | | | | | | | | | | | | | |
| Neogenin | 0 | 0 | 0 | 0 | 0 | 0 | 0 | 7 | 1 | 0 | 0 | 7 | 2 | 0 | 21 |
| IPI00129159.1 | | | | | | | | | | | | | | | |

Experiments were then undertaken to isolate and identify the active protein(s) in the retentate. SDS-PAGE analysis of the active and inactive FPLC fractions from the eEND2 100K retentate and the inactive Fr3 from the 3T3 100K retentate revealed a simple banding pattern in the active eEND2 100K/Fr3 fraction (FIG. 10). The three fractions were then subjected to mass spectroscopic analysis, combined with proteonomic studies and protein comparisons

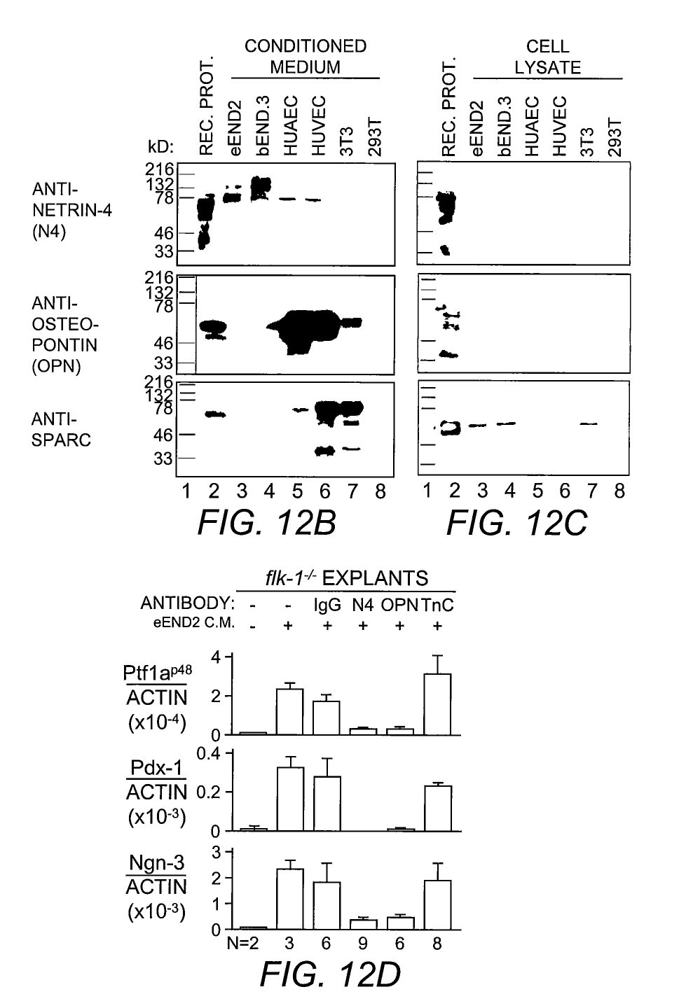

The ability of purified, recombinant candidate proteins to substitute for the activity of the eEND2 conditioned medium was tested using each protein in the 20-50 ng/ml range. A 50 ng exposure level of osteopontin was sufficient to restore ptf1a expression in the flk-1^{-/-} tissue explants of dorsal pancreatic endoderm. Osteopontin showed variable induction activity with Pdx-1 and Ngn-3 (FIG. 12A). Low concentrations of osteopontin slightly enhanced Isl-1 expression (FIG. 12A). Western blot analysis revealed that osteopontin was detected at low levels in the conditioned medium from eEND2 and bEND.3 cells, and abundantly in the medium from HUAEC, HUVEC, and 3T3 control cells, but not in that from 293T cells (FIG. 12B). Moreover, osteopontin was not detected in cell lysates (FIG. 12C).

The antibodies used for Western blotting were then separately added to eEND2 conditioned medium, in order to determine if their respective antigens were necessary for early pancreatic gene induction in the endoderm explant assay. Notably, anti-osteopontin added to the conditioned medium and cultures inhibited the induction of ptf1a, pdx-1, and ngn-3 mRNAs in the explants, whereas comparable amounts of control IgG or antibody to Tenascin C had no effect (FIG. 12D). These data indicate that osteopontin is necessary for the ptf1a inducing activity in the conditioned medium.

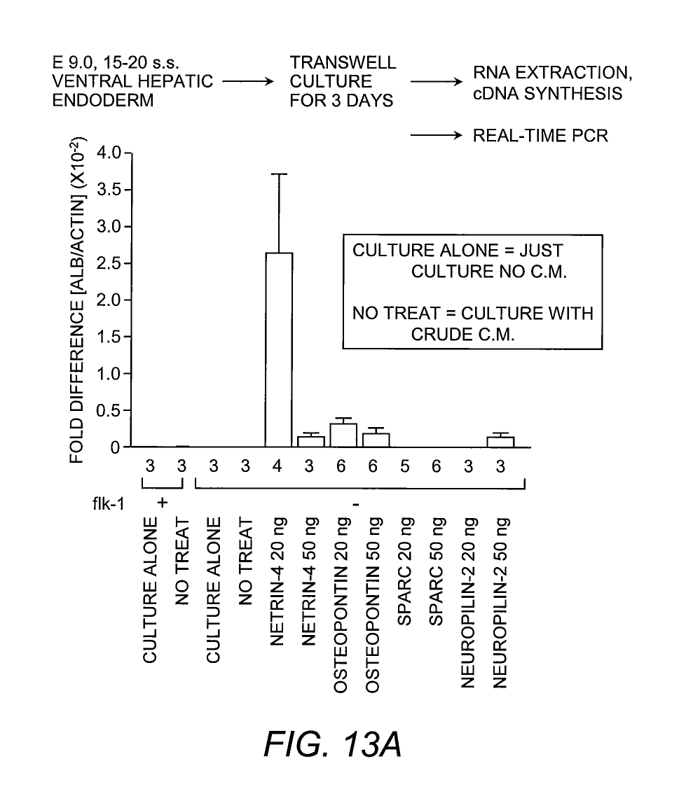

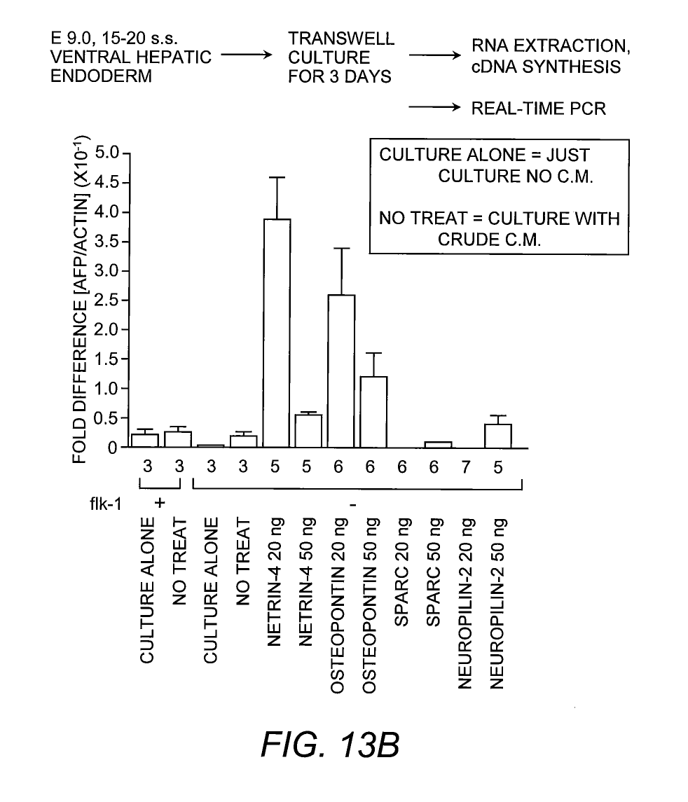

In further experiments, the effect of treatment with osteopontin on induction of albumin and alpha-fetoprotein expression in liver bud explants from flk-1^{-/-} embryos was examined (FIG. 13). It was found that osteopontin treatment produced increases in the expression of these proteins (FIG. 13A, albumin; FIG. 13B, alpha-fetoprotein) in liver bud explants. These data show that osteopontin treatment dramatically enhances liver differentiation of progenitor cells.

Given the results presented herein, particular embodiments of the present invention embrace the use of osteopontin protein, compounds that modulate the activity of osteopontin protein, osteopontin mimetics, or osteopontin agonists to induce the differentiation of endodermal cells, cells derived from embryonic stem cells, other stem and progenitor cells, and liver and pancreatic progenitor cells into pancreatic or liver cells, restore function to damaged liver tissue, or to treat diseases of the liver. Osteopontin protein, as used in the context of the present invention, is intended to include human osteopontin as set forth in SEQ ID NO: 1, as well as homologs, variants or biologically active fragments of osteopontin. A comparative analysis of several osteopontin homologs has been published (Crivello, J. F. and E. Delvin. 1992. J. Bone Min. Res. 7:693-699).

As is conventional in the art, endodermal cells are cells which differentiate into epithelial cells of the pancreas, gut endothelial cells, and hepatocytes. An endodermal cell of the present invention, also commonly referred to as an endodermal progenitor cell, can be obtained using any conventional method known in the art, or alternatively, an endodermal cell can be a endodermal cell line. Moreover, an endodermal cell of the invention can be isolated or be a cell of a tissue explant, i.e., tissue taken from the body and grown in an artificial medium.

Also of use in the instant method are pancreatic and liver progenitor cells as well as cells derived from embryonic stem cells, adult stem cells or other stem or progenitor cells. It is contemplated that such cells can be directly differentiated into pancreatic or liver cells via osteopontin treatment, or alternatively be simultaneously or sequentially exposed to other epigenetic signals that mimic in vivo pancreatic or liver development. For example, when employing embryonic stem cells, said cells can first be contacted with serum, activin and retinoic acid to generate pancreatic endodermal cells (Shim, et al. (2007) Diabetologia, PMID: 17457565) and subsequently matured to pancreatic cells via osteopontin treatment. See also the teachings of Schroeder, et al. ((2006) Nat Protoc. 1(2):495-507) for epigenetic signals which differentiate embryonic stem cells into endodermal cells. Similarly, treatment of embryonic stem cells with human activin A and a deleted variant of hepatocyte growth factor (dHGF) (Chen, et al. (2006) Cell Transplant. 15(10):865-71) in combination with osteopontin can be used to induce differentiation of hepatocytes. The isolation of stem cells and progenitor cells is routinely practiced in the art and any conventional method can be employed to isolate such cells.

Endodermal cells, cells derived from embryonic stem cells, other stem and progenitor cells, and pancreatic and liver progenitor cells of the present invention can be characterized in the following manner: responsiveness to growth factors, specific gene expression, antigenic markers on the surface of such cells, and/or basic morphology. For example, extent of growth factor responsivity, e.g., the concentration range of growth factor to which they will respond to, the maximal and minimal responses, and to what other growth factors and conditions to which they might respond, can be used to characterize the subject endodermal cells. Furthermore, isolated endodermal cells can be identified by the presence or absence of particular markers. By way of illustration, an endodermal progenitor cell can be identified by the expression of markers such as FoxA2 (HNF3 beta).

An endodermal cell, cells derived from embryonic stem cells, other stem or progenitor cells, and pancreatic or liver progenitor cells of the invention can be maintained in tissue culture in vitro or ex vivo. There are a number of suitable tissue culture media that exist for culturing tissue from animals. Some of these are complex and some are simple. While endodermal cells, cells derived from embryonic stem cells, other stem or progenitor cells, and pancreatic and liver progenitor cells can be grown in complex media, it will generally be preferred that the explants be maintained in a simple medium, such as Dulbecco's Minimal Essential Media (DMEM), in order to effect more precise control over the differentiation of the endodermal cell, cells derived from embryonic stem cells, other stem or progenitor cells, and pancreatic and liver progenitor cells into the desired cell. Moreover, when the endodermal cell, other stem and progenitor cells, and pancreatic or liver progenitor cells are of an explant, the explant can be maintained in the absence of sera for extended periods of time. In some embodiments, growth factors or other mitogenic agents are not included in the primary media for maintenance of cell cultures in vitro, but are used subsequently to cause proliferation of distinct populations of cells. Such agents are well-known to those skilled in the art and include, but are not limited to, hepatocyte growth factor (HGF), Epidermal Growth Factor (EGF), Fibroblast Growth Factors (FGF), Keratinocyte growth factor (KGF), and the like.

Endodermal cell, cells derived from an embryonic stem cell, other stem or progenitor cell, and pancreatic or liver progenitor cell cultures can be maintained in any suitable culture vessel, such as a 12- or 24-well microplate, and can be maintained under typical culture conditions for cells isolated from the same animal, e.g., such as 37° C. in 5% CO₂. The cultures can be shaken for improved aeration, the speed of shaking being, for example, 12 rpm.

In other embodiments, endodermal cells, cells derived from embryonic stem cells, other stem or progenitor cells, and pancreatic or liver progenitor cells and/or explants are cultured on feeder layers, e.g., layers of feeder cells which secrete inductive factors or polymeric layers containing inductive factors.

In another embodiment, the subject endodermal cells, cells derived from embryonic stem cells, other stem or progenitor cells, and pancreatic or liver progenitor cells are implanted into one of a number of regeneration models used in the art, e.g., a host animal which has undergone partial pancreatectomy or partial hepatectomy.

In accordance with the present invention, cultured endodermal cells, cells derived from embryonic stem cells, other stem or progenitor cells, and pancreatic or liver progenitor cells or explants containing endodermal cells are contacted with an effective amount of an osteopontin protein, an osteopontin mimetic, or to an osteopontin agonist so that the endodermal cells, cells derived from embryonic stem cells, other stem or progenitor cells, and pancreatic or liver progenitor cells differentiate into pancreatic cells or hepatocytes or bile duct structures. Differentiation in the present context refers to a status of cells in which the cells develop specific morphological or functional properties. Cells may differentiate into a specific tissue or organ. On the other hand, undifferentiated cells are difficult to distinguish each other in a population of cells, since each cell does not have any or little specific morphological or functional properties.

Determination of whether a cell has differentiated into a liver cell can be achieved by the detection of markers specific to these cell types. In an illustrative embodiment, proteins of the hepatocyte nuclear factor (HNF) transcription factor family, e.g., HNF1-4, are known to be expressed in liver progenitors at various times during liver development. For example, the endodermal cell can express FoxA2 (HNF3-beta) and early liver progenitors can express HNF proteins such as HNF1α, HNF2β, HNF3γ, and/or HNF4. The glucose transporter Glut2 is a marker for early pancreatic cells.

In another illustrative embodiment, homeodomain type transcription factors such as STF-1 (also known as IPF-1, IDX-1 or PDX) have been shown to mark different populations of the developing pancreas. Some LIM genes have also been shown to regulate insulin gene expression and would also be markers for protodifferentiated β-islet cells. Likewise, certain of the PAX genes, such as PAX6, are expressed during pancreas formation and can be used to characterize certain pancreatic endodermal cell populations. Other markers of pancreatic endodermal cells include the pancreas-specific transcription factor PTF-1, and hXBP-1 and the like. Moreover, certain of the HNF proteins are expressed during early pancreas development and can used as markers for pancreatic endodermal cells.

Endodermal cells giving rise to pancreatic cells may also express such markers as villin and/or tyrosine hydroxylase, as well as secrete such factors as insulin, glucagon and/or neuropeptide Y.

In other embodiments, differentiated pancreatic cells can be characterized by binding to lectin(s), e.g., to a plant lectin such as peanut agglutinin. In certain embodiments, the lectin is Amaranthus caudatus Lectin (ACL, ACA); Bauhinia purpurea Lectin (BPL, BPA); Concanavalin A (Con A); Succinylated Concanavalin A (Con A); Datura stramonium Lectin (DSL); Erythrina cristagalli Lectin (ECL, ECA); Galanthus nivalis Lectin (GNL); Lens culinaris Agglutinin (LCA); Isolectin-B4; Lycopersicon esculentum (Tomato) Lectin (LEL, TL); Narcissus pseudonarcissus Lectin (NPL, NPA, DL); Peanut Agglutinin (PNA); Phaseolus vulgaris Agglutinin (PHA); Pisum sativum (PSA); Solanum tuberosum (Potato) Lectin (STL, PL); Soybean Agglutinin (SBA); Wheat Germ Agglutinin (WGA); Succinylated Wheat Germ Agglutinin; and the like.

For instance, various components of the human pancreas can be marked by different lectins. DSL marks inter- and intralobular ducts. LCA appears to mark mesenchyme. ECL marks intralobular ducts without marking larger ducts. Succinylated-Wheat Germ Agglutinin marks a subset of main duct cells and is quite restricted compared to WGA.

Endodermal cells giving rise to hepatocytes express markers such as albumin, HNF-4α, α-fetoprotein, transthyretin, and CK-18. Moreover, hepatocytes can be identified based on the development of at least one property of the liver, including but not limited to, regulation of blood sugar; regulation of lipids; regulation of amino acids; production of heat; formation of bile; formation of cholesterol; metabolism of hormones, toxins, etc.; formation of heparin; and storage of vitamins such as vitamin A and D.

Having demonstrated that osteopontin induces differentiation of endodermal cells into pancreatic cells, it is contemplated that an osteopontin protein, as well as osteopontin mimetics, osteopontin agonists, compounds that modulate the activity of osteopontin, or cells differentiated with osteopontin to exhibit pancreatic or liver phenotypes can be used in the treatment of a variety of diseases or conditions. Generally, treatment involves altering pancreatic or liver cell function, improving pancreatic or liver cell function, or replacing damaged pancreatic cells or liver cells to prevent or treat diseases or conditions of the pancreas or liver

In some embodiments, the invention contemplates the in vivo administration of an osteopontin protein or an osteopontin agonist to subjects which have been transplanted with pancreatic tissue, as well as to subjects which have a need for improved pancreatic performance, especially of glucose-dependent insulin secretion. In other embodiments, the invention provides in vitro or ex vivo differentiation of endodermal cells into cells exhibiting a pancreatic phenotype for transplant into subjects which have a need for improved pancreatic performance, especially of glucose-dependent insulin secretion. Accordingly, particular embodiments embrace differentiation of cells into insulin-producing cells, and more desirably, glucose-responsive insulin-producing cells. In still other embodiments, subjects in need of improved liver function or performance are administered an osteopontin protein or cells differentiated with osteopontin to treat diseases or conditions of the liver.

It is contemplated that the cells differentiated in vitro or ex vivo for use in treatment of a subject can be either syngeneic, allogeneic or xenogeneic. Thus, in certain embodiments, small samples of pancreatic or liver tissue from a donor or self can be obtained without sacrificing or seriously injuring the donor. The endodermal cells (e.g., either isolated or as cells of the explant) are subsequently contacted with an osteopontin protein and optionally amplified, and subsequently injected or implanted into a recipient subject, i.e., either self or a suitable recipient. When allogeneic or xenogeneic transplantation is conducted, rejection response may optionally obviated by any method known in the art such as administering immunosuppressive agent (e.g., azathiopurine, cyclophosphamide, etc.).

In accordance with the present invention, treatment involves administration of an effective amount of an osteopontin protein or osteopontin-differentiated endodermal cell, cells derived from embryonic stem cells, other stem and progenitor cells, and liver and pancreatic cells to a subject in need of treatment thereby ameliorating or alleviating at least one sign or symptom of the disease or condition of the subject. Generally, when treatment involves the use of an osteopontin protein or osteopontin mimetics, osteopontin agonists, compounds that modulate the activity of osteopontin, such molecules are formulated into a pharmaceutical composition containing the molecule in admixture with a pharmaceutically acceptable vehicle. For example, the molecule could be formulated in any pharmaceutically acceptable vehicle that would be compatible with the type of cells or tissue being contacted. Formulations of the present invention contemplated would include injectable solutions as well as suitable oral, dermal, intramuscular, or subcutaneous formulations. Contemplated as well are vectors appropriate for delivering nucleic acid encoding an osteopontin protein to the targeted tissue or the targeted cells.

Pharmaceutical compositions can be prepared by methods, and contain vehicles, which are well-known in the art. A generally recognized compendium of such methods and ingredients is Remington: The Science and Practice of Pharmacy, Alfonso R. Gennaro, editor, 20th ed. Lippincott Williams & Wilkins: Philadelphia, Pa., 2000. A pharmaceutically acceptable vehicle, composition or carrier, such as a liquid or solid filler, diluent, excipient, or solvent encapsulating material, is involved in carrying or transporting the subject compound from one organ, or portion of the body, to another organ, or portion of the body. Each vehicle must be acceptable in the sense of being compatible with the other ingredients of the formulation and not injurious to the subject being treated.

Examples of materials which can serve as pharmaceutically acceptable vehicles include sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; pH buffered solutions; polyesters, polycarbonates and/or polyanhydrides; and other non-toxic compatible substances employed in pharmaceutical formulations. Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Pharmaceutical compositions appropriately formulated for parenteral (for example, by intravenous, intraperitoneal, subcutaneous or intramuscular injection), topical (including buccal and sublingual), oral, intranasal, intravaginal, or rectal administration can be prepared according to standard methods.

The selected dosage level will depend upon a variety of factors including the activity of the particular molecule employed, the route of administration, the time of administration, the rate of excretion or metabolism of the particular agent being employed, the duration of the treatment, other drugs, compounds and/or materials used in combination with the particular agent employed, the age, sex, weight, condition, general health and prior medical history of the patient being treated, and like factors well known in the medical arts.

A physician or veterinarian having ordinary skill in the art can readily determine and prescribe the effective amount of the pharmaceutical composition required. For example, the physician or veterinarian could start doses of a molecule at levels lower than that required in order to achieve the desired therapeutic effect and gradually increase the dosage until the desired effect is achieved. In the case of the treatment of cells either in vitro or ex vivo, doses of Osteopontin would be expected to be in the range of nanograms/ml or micrograms/ml.

In embodiments embracing treatment with cells which have been differentiated using an osteopontin protein, common methods of administering such cells to subjects, particularly human subjects, are well-known in the art. Such methods include injection or implantation of the cells into target sites in the subjects using, e.g., a delivery device which facilitates introduction of the cells into the subjects. Such delivery devices include tubes, e.g., catheters, for injecting cells and fluids into the body of a recipient subject. In certain embodiments, the tubes additionally have a needle, e.g., a syringe, through which the cells of the invention can be introduced into the subject at a desired location. The differentiated cells of the invention can be inserted into such a delivery device, e.g., a syringe, in different forms. For example, the cells can be suspended in a solution or embedded in a support matrix when contained in such a delivery device. As used herein, the term “solution” includes a pharmaceutically acceptable vehicle in which the cells of the invention remain viable. The solution is preferably sterile and fluid to the extent that easy syringability exists. Preferably, the solution is stable under the conditions of manufacture and storage and preserved against the contaminating action of microorganisms such as bacteria and fungi through the use of, for example, parabens, chlorobutanol, phenol, ascorbic acid, thimerosal, and the like.

Support matrices in which the differentiated cells can be incorporated or embedded include matrices which are recipient-compatible and which degrade into products which are not harmful to the recipient. Natural and/or synthetic biodegradable matrices are examples of such matrices. Natural biodegradable matrices include plasma clots, e.g., derived from a mammal, and collagen matrices. Synthetic biodegradable matrices include synthetic polymers such as polyanhydrides, polyorthoesters, and polylactic acid. Other examples of synthetic polymers and methods of incorporating or embedding cells into these matrices are known in the art. See, e.g., U.S. Pat. No. 4,298,002 and U.S. Pat. No. 5,308,701. These matrices provide support and protection for the fragile differentiated cells in vivo and are, therefore, a desired form in which the differentiated cells are introduced into the recipient subjects.

The present invention also provides substantially pure differentiated cells which can be used therapeutically for treatment of various disorders associated with insufficient functioning of the pancreas or liver.

To illustrate, the subject differentiated cells can be used in the treatment or prophylaxis of a variety of pancreatic disorders, both exocrine and endocrine. For instance, the differentiated cells can be used to repair a partial pancreatectomy, e.g., excision of a portion of the pancreas. Likewise, such cell populations can be used to regenerate or replace pancreatic tissue loss due to, pancreatolysis, e.g., destruction of pancreatic tissue, such as pancreatitis, i.e., a condition due to autolysis of pancreatic tissue caused by escape of enzymes into the substance.

In an exemplary embodiment, the subject differentiated cells can be provided to patients suffering from any insulin-deficiency disorder such as diabetes. Diabetes is characterized by pancreatic islet destruction or dysfunction leading to loss of glucose control. Diabetes mellitus is a metabolic disorder defined by the presence of chronically elevated levels of blood glucose (hyperglycemia). Insulin-dependent (Type 1) diabetes mellitus (IDDM) results from an autoimmune-mediated destruction of the pancreatic β-cells with consequent loss of insulin production, which results in hyperglycemia. Type 1 diabetics require insulin replacement therapy to ensure survival. Non-insulin-dependent (Type 2) diabetes mellitus (NIDDM) is initially characterized by hyperglycemia in the presence of higher-than-normal levels of plasma insulin (hyperinsulinemia). In Type 2 diabetes, tissue processes which control carbohydrate metabolism are believed to have decreased sensitivity to insulin. Progression of the Type 2 diabetic state is associated with increasing concentrations of blood glucose, and coupled with a relative decrease in the rate of glucose-induced insulin secretion.

The primary aim of treatment in both forms of diabetes mellitus is the same, namely, the reduction of blood glucose levels to as near normal as possible. Treatment of Type 1 diabetes involves administration of replacement doses of insulin. In contrast, treatment of Type 2 diabetes frequently does not require administration of insulin. For example, initial therapy of Type 2 diabetes may be based on diet and lifestyle changes augmented by therapy with oral hypoglycemic agents such as sulfonylurea. Insulin therapy may be required, however, especially in the later stages of the disease, to produce control of hyperglycemia in an attempt to minimize complications of the disease, which may arise from islet exhaustion.

Tissue-engineering approaches have also been employed, wherein treatment has focused on transplanting healthy pancreatic islets, usually encapsulated in a membrane to avoid immune rejection. Three general approaches have been tested in animal models. In the first, a tubular membrane is coiled in a housing that contained islets. The membrane is connected to a polymer graph that in turn connects the device to blood vessels. By manipulation of the membrane permeability, so as to allow free diffusion of glucose and insulin back and forth through the membrane, yet block passage of antibodies and lymphocytes, normoglycemia was maintained in pancreatectomized animals treated with this device (Sullivan et al. (1991) Science 252:718).

In a second approach, hollow fibers containing islet cells were immobilized in the polysaccharide alginate. When the device was place intraperitoneally in diabetic animals, blood glucose levels were lowered and good tissue compatibility was observed (Lacey et al. (1991) Science 254:1782).

Finally, islets have been placed in microcapsules composed of alginate or polyacrylates. In some cases, animals treated with these microcapsules maintained normoglycemia for over two years (Lim et al. (1980) Science 210:908; O′Shea et al. (1984) Biochim. Biochys. Acta. 840:133; Sugamori et al. (1989) Trans. Am. Soc. Artif Intern. Organs 35:791; Levesque et al. (1992) Endocrinology 130:644; and Lim et al. (1992) Transplantation 53:1180). However, all of these transplantation strategies require a large, reliable source of donor islets.

Differentiation of cells in accordance with the present invention can be used for treatment of diabetes because endodermal cells can be differentiated into cells of pancreatic lineage, e.g., β-islet cells. Endodermal cells, cells derived from embryonic stem cells, other stem and progenitor cells, and liver and pancreatic progenitor cells can be cultured in vitro in the presence of osteopontin and under conditions which can further induce these cells to differentiate into mature pancreatic cells, or they can undergo differentiation in vivo once introduced into a subject. Many methods for encapsulating cells are known in the art. For example, a source of β-islet cells producing insulin is encapsulated in implantable hollow fibers. Such fibers can be pre-spun and subsequently loaded with the β-islet cells (U.S. Pat. No. 4,892,538; U.S. Pat. No. 5,106,627; Hoffman et al. (1990) Expt. Neurobiol. 110:39-44; Jaeger et al. (1990) Prog. Brain Res. 82:41-46; and Aebischer et al. (1991) J. Biomech. Eng. 113:178-183), or can be co-extruded with a polymer which acts to form a polymeric coat about the β-islet cells (U.S. Pat. No. 4,391,909; U.S. Pat. No. 4,353,888; Sugamori et al. (1989) Trans. Am. Artif. Intern. Organs 35:791-799; Sefton et al. (1987) Biotechnol. Bioeng. 29:1135-1143; and Aebischer et al. (1991) Biomaterials 12:50-55).

Moreover, in addition to providing a source of implantable cells, either in the form of the progenitor cell population or the differentiated progeny thereof, the subject cells can be used to produce cultures of pancreatic cells for production and purification of secreted factors. For instance, cultured cells can be provided as a source of insulin. Likewise, exocrine cultures can be provided as a source for pancreatin.

Likewise, it is contemplated that differentiation of cells in accordance with the present invention can be used for treatment of hepatic diseases, disorders or conditions including but not limited to: alcoholic liver disease, hepatitis (A, B, C, D, etc.), focal liver lesions, primary hepatocellular carcinoma, large cystic lesions of the liver, focal nodular hyperplasia granulomatous liver disease, hepatic granulomas, hemochromatosis such as hereditary hemochromatosis, iron overload syndromes, acute fatty liver, hyperemesis gravidarum, intercurrent liver disease during pregnancy, intrahepatic cholestasis, liver failure, fulminant hepatic failure, jaundice or asymptomatic hyperbilirubinemia, injury to hepatocytes, Crigler-Najjar syndrome, Wilson's disease, alpha-1-antitrypsin deficiency, Gilbert's syndrome, hyperbilirubinemia, nonalcoholic steatohepatitis, porphyrias, noncirrhotic portal hypertension, noncirrhotic portal hypertension, portal fibrosis, schistosomiasis, primary biliary cirrhosis, Budd-Chiari syndrome, hepatic veno-occlusive disease following bone marrow transplantation, etc.

Yet another aspect of the present invention provides methods for screening various compounds for their ability to modulate growth, proliferation or differentiation of distinct endodermal cell populations. In an illustrative embodiment, the subject endodermal cells, and their differentiated progeny, can be used to screen various compounds or natural products. Such cells can be maintained in minimal culture media for extended periods of time (e.g., for 7-21 days or longer) and can be contacted with any compound, e.g., small molecule or natural product, e.g., growth factor, to determine the effect of such compound on cellular growth, proliferation or differentiation of the endodermal cells. Detection and quantification of growth, proliferation or differentiation of these cells in response to a given compound provides a means for determining the compound's efficacy at inducing one of the growth, proliferation or differentiation in a given cell type. Methods of measuring cell proliferation are well-known in the art and most commonly include determining DNA synthesis characteristic of cell replication. There are numerous methods in the art for measuring DNA synthesis, any of which may be used according to the invention. In an embodiment of the invention, DNA synthesis can be determined using a radioactive label (3H-thymidine) or labeled nucleotide analogues (BrdU) for detection by immunofluorescence. The efficacy of the compound can be assessed by generating dose response curves from data obtained using various concentrations of the compound. A control assay can also be performed to provide a baseline for comparison. Identification of the endodermal cell population(s) amplified in response to a given test agent can be carried out according to such phenotyping as described above.

The following non-limiting examples are provided to further illustrate the present invention.

### EXAMPLES

### Example 1

### Liver and Pancreatic Endoderm Dissection and Transwell Culture

The purpose of this explant system is to support the morphogenetic changes of liver or pancreatic bud into highly differentiated structure. This system allows tissue to grow 3-dimensionally, and allows for examination of morphological changes of specific cell domains in vitro, rather than only detection of the expression of specific genes in explants.

a) Preparation

Culture medium was prepared. Dulbecco's modified Eagle medium containing 10% calf serum (Hyclone), penicillin (100 unit/ml)/streptomycin (1000 g/ml) was used as culture media, also containing 0.2 Matrigel (Collaborative Biomedical Products, Becton Dickinson). Transwell culture plates (Corning; 12 mm membrane diameter and 3.0 micrometer pore size) were used. The upper chambers of the plates were coated with 400 microliters of collagen substrata containing 96.3 microgram/ml of Collagen Type 1 (BD Biosciences) in 0.02 N acetic acid/phosphate-buffered saline (PBS) at 37° C. for at least for 1 hour. Then the solution was aspirated and the upper chambers were washed twice with pre-warmed PBS and once with medium. Just before starting tissue culture, the medium was aspirated from the upper chamber and 400 microliters/well of culture medium with 0.2% matrigel was replaces in the upper chambers, with 600 microliters/well of the same medium being places in the lower chambers.

b) Dissection of Foregut Endoderm

Liver and dorsal pancreatic bud region can be recognized morphologically after E9.0. For the studies of morphological changes in liver and dorsal pancreatic bud, embryos from E9.0-10.0 were used and cultured onto the Transwell plates as described above.

Noon of the day of vaginal plug discovery was identified as E0.5. Embryos were removed from uteri at appropriate times, transferred to dishes containing PBS and 0.1% BSA, and dissected free from decidual tissues. The embryos were then transfed to black wax dissecting dishes containing a few drops of PBS with 0.1% BSA.

The yolk sac was then carefully removed under a dissecting microscope, using electrolytically etched tangusten needles. The cardiac tube and midgut/hindgut below the liver and dorsal pancreatic bud were then removed, so that the midsection could be obtained. By changing the direction of the midsection, the gut tube was recognized. The liver and dorsal pancreatic bud regions were then cut from the gut tube. After cleaning away extra tissue, the explants were transferred to the upper chambers of the Transwell plates.

c) Tissue Culture

The explants were incubated in 5% CO₂/95% air at 37° C. for 1-3 days and subjected to further experiments. Under a microscope, the presence of cardiac mesodermal cells in the explants were recognized as beating cells. The growth of explants were recorded with a phase contrast microscope.

After culturing, the explants were subjected to RNA extraction for RT-PCR (real-time PCR), in situ hybridization, or immunohistochemistry. For in situ hybridization and immunohistochemistry, tissues were fixed on the slide in 4% paraformaldehyde in PBS for a few hours to overnight at 4° C., then dehydrated with a series of methanol washes. The explants were stored at −20° C. for several months.

What is claimed is:

1. A method for inducing differentiation of an endodermal cell or a progenitor cell into a pancreatic or liver cell comprising contacting an endodermal cell or a progenitor cell with an effective amount of osteopontin thereby inducing differentiation of the endodermal cell or the progenitor cell into a pancreatic or liver cell.

2. The method of claim 1 wherein said cell is a partially differentiated liver or pancreatic progenitor cell.

3. A method for altering liver cell function comprising contacting a liver cell with an effective amount of osteopontin, wherein contact of the cell with osteopontin results in an alteration in the function of the liver cell.

4. A method for altering liver cell function comprising contacting a liver cell with an effective amount of a composition comprising an osteopontin protein, an osteopontin antibody, an osteopontin mimetic, an osteopontin agonist, an osteopontin antagonist, a mutated osteopontin protein, or an osteopontin variant or fragment thereof, formulated in a pharmaceutically acceptable vehicle, wherein contact of the cell with said composition results in an alteration in the function of the liver cell.

5. A method for preventing or treating a disease of the liver in a patient comprising administering to a patient a therapeutically effective amount of a composition comprising osteopontin formulated in a pharmaceutically acceptable vehicle, wherein administration of said composition results in prevention or treatment of a disease of the liver.

6. A method for preventing or treating a disease of the liver in a patient comprising administering to a patient a therapeutically effective amount of a composition comprising an osteopontin protein, an osteopontin antibody, an osteopontin mimetic, an osteopontin agonist, an osteopontin antagonist, a mutated osteopontin protein, or an osteopontin variant or fragment thereof, formulated in a pharmaceutically acceptable vehicle, wherein administration of the composition results in prevention or treatment of a disease of the liver.

7. A method of restoring function of a damaged liver tissue which comprises contacting a damaged liver tissue with an effective amount of osteopontin, wherein contact of the damaged tissue with osteopontin results in a restoration of function of the damaged tissue.

8. A composition comprising an isolated pancreatic or liver cell differentiated by the method of claim 1.

9. A pharmaceutical composition comprising isolated pancreatic or liver cells differentiated by the method of claim 1 and a pharmaceutically acceptable vehicle.
